(19) United States
(12) Patent Application Publication
(10) Pub. No.: US 2014/0378426 A1
(43) Pub. Date: Dec. 25, 2014

(54) Methods and compositions for enhancing visual function

(71) Applicant: Anne Hanneken, La Jolla, CA (US)

(72) Inventor: Anne Hanneken, La Jolla, CA (US)

(21) Appl. No.: 14/120,727

(22) Filed: Jun. 20, 2014

Related U.S. Application Data
(60) Provisional application No. 61/837,348, filed on Jun. 20, 2013.

(51) Int. Cl. A61K 31/565; A61K 9/00

(52) U.S. Cl. CPC A61K 31/565; A61K 9/0048
USPC 514/182

(57) ABSTRACT

The invention provides compositions of and methods for using estradiol and related compounds to improve visual function and to treat ocular degenerative disorders.

### CROSS-REFERENCE TO RELATED APPLICATIONS

The subject patent application claims the benefit of priority to U.S. Provisional Patent Application No. 61/837,348 (filed Jun. 20, 2013). The full disclosure of the priority application is incorporated herein by reference in its entirety and for all purposes.

### STATEMENT OF GOVERNMENT SUPPORT

This invention was made with government support under Grant No. TR000109 awarded by the National Institutes of Health. The U.S. Government therefore has certain rights in the invention.

### BACKGROUND OF THE INVENTION

Retinal degenerative disorders such as age-related macular degeneration (AMD) and diabetic retinopathy (DR) are prevalent ocular disorders in industrialized nations. For example, AMD is the most common cause of visual loss in patients over the age of 55 years in the United States and other developed countries. The etiology is multifactorial, linked to complex environmental factors, smoking and vascular disease, and genetic polymorphisms involving the innate immune system. Ninety percent of patients suffer from the dry form of the disease, which is characterized by the degeneration of retinal pigment epithelial cells (RPE) and the loss of photoreceptors in the outer retina. The dry or atrophic form of the disease is characterized by the accumulation of drusen, thickening of Bruch's membrane, atrophy of retinal pigment epithelial cells and ultimately photoreceptor degeneration. As the disease progresses, patients complain of poor visual sensitivity, poor dark adaptation and decreased visual discrimination.

While currently there are some regimens for slowing vision loss in patients with such ocular disorders, there are still no effective treatments to reverse the progression of these retinal degenerative diseases. For example, patients with dry AMD are managed with nutritional supplements, which have been shown to reduce the progression of visual loss by 30%. Unfortunately, there is no treatment to improve vision in many retinal degenerative diseases (e.g., dry AMD). No medication has been developed to enhance visual transduction in patients with such disorders.

Thus, there is a need in the art for better means for preserving and improving vision in subjects having or at risk of developing various retinal degenerative disorders. The present invention is directed to this and other needs.

### SUMMARY OF THE INVENTION

In one aspect, the invention provides methods of enhancing visual function and improving vision in a subject. The methods entail administering to one or more eyes of the subject a pharmaceutical composition that contains a therapeutically effective dose of an estradiol or derivative compound. In some embodiments, the administered estradiol or derivative compound is 17β-estradiol. In some embodiments, the estradiol or derivative compound is locally administered to the eyes, e.g., via eye drops. Typically, the administered pharmaceutical composition can additionally contain a pharmaceutically acceptable carrier.

Some methods of the invention are directed to treating a subject afflicted with or at risk of developing an ocular degenerative disorder, e.g., a retinal degenerative disorder or ocular vascular disorder. In some embodiments, the subject to be treated has or is at risk of developing age-related macular degeneration (AMD).

In a related aspect, the invention provides methods of preserving vision in the eyes of a subject. The methods involve prophylactically administering to one or more eyes of the subject a pharmaceutical composition that contains a therapeutically effective amount of an estradiol or derivative compound. In some preferred embodiments, the administered compound is 17β-estradiol. In some preferred embodiments, the compound is administered to the eyes of the subject via a local route, e.g., in eye drops. The subject can be a normal aging person or one who is afflicted with or at risk of developing an ocular degenerative disorder (e.g., age-related macular degeneration).

In another related aspect, the invention provides methods for improving or preserving vision of a subject afflicted with or at risk of developing age-related macular degeneration (AMD). The methods entail administering to the subject a pharmaceutical composition that contains a therapeutically effective dose of an estradiol or derivative compound. In some preferred embodiments, the administered compound is 17β-estradiol. In some preferred embodiments, the compound is administered to the eyes of the subject via a local route, e.g., in eye drops. In various embodiments, the administered pharmaceutical composition can further contain a pharmaceutically acceptable carrier. In some embodiments, the subject to be treated has or is at risk of developing the dry form of AMD.

In another aspect, the invention provides methods for treating or ameliorating a symptom associated with age-related macular degeneration (AMD) in a subject. The methods involve administering to the subject a pharmaceutical composition that contains a therapeutically effective dose of an estradiol or derivative compound. In some embodiments, the administered compound is 17β-estradiol. In some embodiments, the estradiol or derivative compound is administered to one or more eyes of the subject via a local route of administration, e.g., in eye drops. Some preferred embodiments of the invention are directed to treating or ameliorating symptoms in a subject afflicted with dry AMD. Examples of symptoms that can be treated include impaired central vision, diminished night vision, impaired contrast sensitivity, distorted vision or blurred vision.

Another aspect of the invention relates to kits or pharmaceutical combinations for carrying out the therapeutic applications of the invention. The kits typically include a pharmaceutical composition containing a therapeutically effective amount of an estradiol or derivative compound (e.g., 17β-estradiol), a device for applying the composition, and an instruction detailing the use of the composition for improving visual function in a subject. In some embodiments, the pharmaceutical composition is a topical eye drop formulation, and the device is an eye drop dispenser.

A further understanding of the nature and advantages of the present invention may be realized by reference to the remaining portions of the specification and claims.

### BRIEF DESCRIPTION OF THE DRAWINGS

FIG. 1 shows that estradiol enhances opsin signaling and transducin activation in visual phototransduction cascade.

### DETAILED DESCRIPTION OF THE INVENTION

### I. Overview

The present invention relates to methods and composition for improving visual function, esp. in subjects afflicted or at risk of developing ocular disorders such as age-related macular degeneration. The invention is predicated in part on the discoveries by the present inventor that activation of transducin in phototransduction cascade can be enhanced in vitro with estradiol related compounds. These findings indicate that, by increasing opsin signaling, such compounds could also promote visual function. Specifically, the inventor observed that 17β-estradiol enhanced rhodopsin signaling in biochemical assays, likely as an allosteric modulator of opsin signaling. The inventor's observation suggests that allosteric modulators of opsin signaling could be important for highly efficient visual transduction, and that the typical age-dependent decrease of circulating steroid hormones could have a negative impact on vision, which would be magnified in the setting of a degenerative retinal disease. It further suggests that visual sensitivity can be improved in aging subjects or subjects with age-related vision deterioration (e.g., age related macular degeneration) using 17β-estradiol or related analog compounds via potentiating rhodopsin signaling.

In accordance with the inventor's observations, the present invention provides methods for maintaining or enhancing visual function in subjects with visual difficulties that are a result of retinal aging or retinal degenerations. In related embodiments, the invention provides methods for preventing or alleviating vision deterioration in subjects who are at risk of or predispositioned to developing degenerative retinal disorders such as AMD or methods for delaying progression of such disorders. The therapeutic methods of the invention typically involve administering the subjects an effective amount of 17β-estradiol or a derivative compound, e.g., via topical formulations or other routes of administration. Further provided in the invention are pharmaceutical combinations or therapeutic kits for carrying out the various therapeutic applications of the invention.

The following sections provide more detailed guidance for practicing the invention. Although any methods and materials similar or equivalent to those described herein can be used in the practice for testing of the present invention, the preferred materials and methods are described herein.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Academic Press Dictionary of Science and Technology, Morris (Ed.), Academic Press (1^{st} ed., 1992); Illustrated Dictionary of Immunology, Cruse (Ed.), CRC Pr I LLC (2^{nd} ed., 2002); Oxford Dictionary of Biochemistry and Molecular Biology, Smith et al. (Eds.), Oxford University Press (revised ed., 2000); Encyclopaedic Dictionary of Chemistry, Kumar (Ed.), Anmol. Publications Pvt. Ltd. (2002); Dictionary of Microbiology and Molecular Biology, Singleton et al. (Eds.), John Wiley & Sons (3^{rd} ed., 2002); Dictionary of Chemistry, Hunt (Ed.), Routledge (1^{st} ed., 1999); Dictionary of Pharmaceutical Medicine, Nahler (Ed.), Springer-Verlag Telos (1994); Dictionary of Organic Chemistry, Kumar and Anandand (Eds.), Anmol Publications Pvt. Ltd. (2002); and A Dictionary of Biology (Oxford Paperback Reference), Martin and Hine (Eds.), Oxford University Press (4^{th} ed., 2000). In addition, the following definitions are provided to assist the reader in the practice of the invention.

Rhodopsin, also known as visual purple, is a biological pigment in photoreceptor cells of the retina that is responsible for the first events in the perception of light. Rhodopsin is bound to the plasma membrane of the rod and forms transmembrane protein complexes within it. Rhodopsin has two components: rod opsin (or scotopsin), a protein moiety; and 11-cis-retinal, a carotene derivative. When combined, these two subunits create the conjugated rhodopsin molecule. Rhodopsin undergoes a cyclic decomposition and reconstitution in response to the presence of light. This rather complicated cycle is the basis for absorption of light and its transduction into a nervous signal.

Opsins are a group of light-sensitive 35-55 kDa membrane-bound G protein-coupled receptors of the retinylidene protein family found in photoreceptor cells of the retina. Five classical groups of opsins are involved in vision, mediating the conversion of a photon of light into an electrochemical signal, the first step in the visual transduction cascade. Another opsin found in the mammalian retina, melanopsin, is involved in circadian rhythms and pupillary reflex but not in image-forming. Vertebrate opsins can be further subdivided into rod opsins and four types of cone opsin, based on differential spatial expression, spectral sensitivity, and evolutionary history. Rod opsins (rhodopsins, usually denoted Rh), are used in night vision, are thermally stable, and are found in the rod photoreceptor cells. Cone opsins, employed in color vision, are less-stable opsins located in the cone photoreceptor cells. Cone opsins are further subdivided according to their absorption maxima (λmax), the wavelength at which the highest light absorption is observed. Evolutionary relationships, deduced using the amino acid sequence of the opsins, are also frequently used to categorize cone opsins into their respective group. Both methods predict four general cone opsin groups in addition to rhodopsin.

Several closely related opsins exist that differ only in a few amino acids and in the wavelengths of light that they absorb most strongly. Humans have four different other opsins besides the scotopsin component of rhodopsin. The photopsins are found in the different types of the cone cells of the retina and are the basis of color vision. They have absorption maxima for yellowish-green (photopsin I), green (photopsin II), and bluish-violet (photopsin III) wavelengths of light. The remaining opsin (melanopsin) is found in photosensitive ganglion cells and absorbs blue light most strongly.

As used herein, ocular degenerative diseases or disorders broadly encompass any ocular vascular diseases and ocular degenerative disorders that affect the various parts of the eye, including the retina, cornea, iris and macula. While sharing a common prognosis of a progressive loss of vision, these diseases can be inherited, associated with another medical condition, caused by a virus and cancer. Thus, ocular degenerative diseases or disorders include various ocular vascular disorders (e.g., corneal neovascularization or retinal neovascularization), retinal degenerative diseases (e.g., atrophic macular degeneration), retina edema (including macular edema), ischemic retinopathies, vascular hemorrhages, vascular leakage, choroidopathies, retinal injuries and retinal defects involving an interruption in or degradation of the retinal vasculature. Specific examples of such diseases include age related macular degeneration (ARMD or AMD), diabetic retinopathy (DR), presumed ocular histoplasmosis (POHS), retinopathy of prematurity (ROP), sickle cell anemia, and retinitis pigmentosa, as well as retinal injuries. Other examples include rod-cone dystrophies, genetic retinal degenerations (e.g., Lebers Congenital Amaurosis), and other retinal degenerations and/or dystrophies (e.g., Stargardt's disease).

Ocular vascular disorders refer to pathological conditions characterized by altered or unregulated proliferation and invasion of new blood vessels into the structures of ocular tissues such as the retina. Examples of ocular vascular diseases include ischemic retinopathy, iris neovascularization, intraocular neovascularization, age-related macular degeneration, corneal neovascularization, retinal neovascularization, choroidal neovascularization, diabetic retinal ischemia, retinal degeneration and diabetic retinopathy.

Retinopathy of prematurity (ROP) is a disease of the eye that affects prematurely born babies. It is thought to be caused by disorganized growth of retinal blood vessels which may result in scarring and retinal detachment. ROP can be mild and may resolve spontaneously, but may lead to blindness in serious cases. As such, all preterm babies are at risk for ROP, and very low birth weight is an additional risk factor. Both oxygen toxicity and relative hypoxia can contribute to the development of ROP.

Macular degeneration is a medical condition predominantly found in elderly adults in which the center of the inner lining of the eye, known as the macula area of the retina, suffers thinning, atrophy, and in some cases, bleeding. This can result in loss of central vision, which entails inability to see fine details, to read, or to recognize faces. According to the American Academy of Ophthalmology, it is the leading cause of central vision loss (blindness) in the United States today for those over the age of fifty years. Although some macular dystrophies that affect younger individuals are sometimes referred to as macular degeneration, the term generally refers to age-related macular degeneration (AMD or ARMD).

Age-related macular degeneration begins with characteristic yellow deposits in the macula (central area of the retina which provides detailed central vision, called fovea) called drusen between the retinal pigment epithelium and the underlying choroid. Most people with these early changes (referred to as age-related maculopathy) have good vision. People with drusen can go on to develop advanced AMD. The risk is considerably higher when the drusen are large and numerous and associated with disturbance in the pigmented cell layer under the macula. Large and soft drusen are related to elevated cholesterol deposits and may respond to cholesterol lowering agents or the Rheo Procedure.

Advanced AMD, which is responsible for profound vision loss, has two forms: dry and wet. Neovascular or exudative AMD, the “wet” form of advanced AMD, causes vision loss due to abnormal blood vessel growth (choroidal neovascularization) in the choriocapillaris, through Bruch's membrane, ultimately leading to blood and protein leakage below the macula. Central geographic atrophy, the dry form of advanced AMD, results from atrophy to the retinal pigment epithelial layer below the retina, which causes vision loss through loss of photoreceptors (rods and cones) in the central part of the eye. While no treatment is available for this condition, vitamin supplements with high doses of antioxidants, lutein and zeaxanthin, have been demonstrated by the National Eye Institute and others to slow the progression of dry macular degeneration and in some patients, improve visual acuity.

Retinitis pigmentosa (RP) is a group of genetic eye conditions. In the progression of symptoms for RP, night blindness generally precedes tunnel vision by years or even decades. Many people with RP do not become legally blind until their 40s or 50s and retain some sight all their life. Others go completely blind from RP, in some cases as early as childhood. Progression of RP is different in each case. RP is a type of hereditary retinal dystrophy, a group of inherited disorders in which abnormalities of the photoreceptors (rods and cones) or the retinal pigment epithelium (RPE) of the retina lead to progressive visual loss. Affected individuals first experience defective dark adaptation or nyctalopia (night blindness), followed by reduction of the peripheral visual field (known as tunnel vision) and, sometimes, loss of central vision late in the course of the disease.

Macular edema occurs when fluid and protein deposits collect on or under the macula of the eye, a yellow central area of the retina, causing it to thicken and swell. The swelling may distort a person's central vision, as the macula is near the center of the retina at the back of the eyeball. This area holds tightly packed cones that provide sharp, clear central vision to enable a person to see form, color, and detail that is directly in the line of sight. Cystoid macular edema is a type of macular edema that includes cyst formation.

The terms “subject” and “patient” are used interchangeably and refer to mammals such as human patients and non-human primates, as well as experimental animals such as rabbits, rats, and mice, and other animals. Animals include all vertebrates, e.g., mammals and non-mammals, such as dogs, cats, sheeps, cows, pigs, rabbits, chickens, and etc. Preferred subjects for practicing the therapeutic methods of present invention are human. Subjects in need of treatment include patients already suffering from an ocular degenerative disease or disorder as well as those prone to developing the disorder.

As used herein, “treating” or “ameliorating” includes (i) preventing a pathologic condition (e.g., macular degeneration) from occurring (e.g. prophylaxis); (ii) inhibiting the pathologic condition (e.g., macular degeneration) or arresting its development; and (iii) relieving symptoms associated with the pathologic condition (e.g., vision loss in macular degeneration). Thus, “treatment” includes the administration of a pharmaceutical composition of the invention and/or other therapeutic compositions or agents to prevent or delay the onset of the symptoms, complications, or biochemical indicia of an ocular disease described herein, alleviating or ameliorating the symptoms or arresting or inhibiting further development of the disease, condition, or disorder. “Treatment” further refers to any indicia of success in the treatment or amelioration or prevention of the ocular disease, condition, or disorder described herein, including any objective or subjective parameter such as abatement; remission; diminishing of symptoms or making the disease condition more tolerable to the patient; slowing in the rate of degeneration or decline; or making the final point of degeneration less debilitating. Detailed procedures for the treatment or amelioration of an ocular disorder or symptoms thereof can be based on objective or subjective parameters, including the results of an examination by a physician.

Visual function enhancement is defined as an improvement in one or more visual and psychophysical tests that include, but are not restricted to, dark adaptation, contrast sensitivity, macular stress testing, visual sensitivity, reading speed and print size, Snellen and/or ETDRS visual acuity measurements and electroretinogram (ERG) studies. Activity of a compound in enhancing visual function can also be examined via biological or biochemical assays routinely practiced in the art. For example, compounds can be assessed for ability to promote visual signal transduction in a radioactive filter binding assay or a fluorescent assay for rhodopsin activation of transducing signaling. Rhodopsin, upon activation by light, transduces the photon signal by activation of the G-protein, transducin. Transducin is a very important G-protein in vertebrate phototransduction. Heterotrimeric transducin is activated by metarhodopsin II, a conformational change in rhodopsin caused by the absorption of a photon by the rhodopsin moiety retinal. Transducin activation ultimately results in stimulation of the biological effector molecule cGMP phosphodiesterase (PDE) in the phototransduction cascade.

### III. Estradiol Promoting Rhodopsin Mediated Phototransduction

The present inventor observed that estradiol was able to enhance rhodopsin activation of rod transducing, which plays an important role in visual signal transduction (visual phototransduction) cascade. Human visual pigments are composed of four related G-protein coupled receptors (opsins) and a single chromophore, 11-cis retinal that binds to the opsins. In the presence of light, 11-cis retinal isomerizes to all-trans retinal, which leads to conformational changes that open a binding site for transducin and stimulates visual transduction.

The rhodopsin signaling cycle starts when energy from impinging light excites the electrons in the 11-cis-retinal subunit and converts it to a different configuration, 11-trans-retinal. Because this is conformationally incompatible with the scotopsin moiety, it begins to detach from it, and the rhodopsin conjugate begins to break up into its component parts. The disintegration of rhodopsin into retinal and scotopsin is progressive, with a series of short-lived intermediate compounds formed, as shown in the diagram to the right. The eventual result is release of the two components of rhodopsin from each other completely. One of the breakdown products, metarhodopsin II, is the agent that ultimately effects the change in the rod membrane's charge. Metarhodopsin II is an enzyme. It acts to activate a second membrane-bound protein in the rod, transducin. Transducin contains two subunits, Tα and Tβγ, which are made of three polypeptide chains α, β and γ. It is naturally expressed in vertebrate retina rods and cones, with different a subunits in rod and cone photoreceptors. When metarhodopsin activates transducin, the GDP bound to the a subunit (Tα) is exchanged for GTP from the cytoplasm. The a subunit dissociates from the βγ subunits (Tβγ) Activated transducin α-subunit activates cGMP phosphodiesterase (PDE). This enzyme breaks down cGMP, an intracellular second messenger which opens cGMP-gated cation channels. Decrease in cGMP concentration leads to decreased opening of cation channels and subsequently, hyperpolarization of the membrane potential.

Obviously, the rhodopsin has to be reconstituted, or the ability to respond to light will be lost completely in a few seconds at most. This takes place by two side pathways. First, the 11-trans-retinal is re-converted to the 11-cis-retinal form via an isomerase enzyme. Since the scotopsin moiety is present (having been removed from the rhodopsin), it immediately will combine with this to regenerate new rhodopsin. New 11-cis-retinal can also be generated from 11-trans-retinol, or vitamin A. Vitamin A is a derivative form of 11-trans-retinal, enzymatically convertible to it. The isomerase reaction can in turn convert the trans form to the cis isomer, making new 11-cis-retinal available to recombine with scotopsin. By this pathway additional rhodopsin is manufactured to adapt to continuously dark conditions. The pigment epithelium layer of the retina is a storage site for vitamin A.

As detailed in the Examples below, it was found that the presence of 17β-estradiol significantly up-regulated rhodopsin activation of rod transducin. This demonstrates that 17β-estradiol can have broad utility in enhancing visual function by stimulating the phototransduction cascade. In addition to estradiol, various estradiol analog or derivative compounds can also be employed in the practice of the present invention. These estradiol analogs or derivatives should have similar or improved vision-enhancing activity or pharmaceutical properties relative to that of estradiol.

Vision enhancing activity of the estradiol analogs or derivatives can be examined in accordance with methods described herein or well known in the art. For example, the compounds can be assessed for ability to promote rhodopsin activation of rod transducin using a well-described radioactive filter binding assay. Detailed procedures for carrying out the assay are described in the art, e.g., Kono et al., Biochem. 44:799, 2005; Kono et al., FEBS Lett. 580:229-232, 2006; and Robinson et al., Methods Enzymol. 315:207-218, 2000). Transducin can be purified from bovine retina according to the procedure of Wessling-Resnick and Johnson (J. Biol. Chem. 262:12444-7, 1987) and then subjected to ion-exchange chromatography on DE-52. The protein can be dialyzed against 10 mM Tris buffer (pH 7.5) containing 50% (v/v) glycerol and 2 mM MgCl2 and then stored at −20° C. Rhodopsin can be purified from membrane fractions isolated from bovine rod outer segments. Effect of estradiol or an analog compound on rhodopsin activation of rod transducing can then be measured by monitoring binding of [35S]GTPγS to transducin in the presence or absence of the compound. The reaction mixture can contain, e.g., an appropriate buffer (e.g., an HEPES buffer or 10 mM Tris/1 mM MES buffer (pH 7.5)), 1 mM dithiothreitol, 5 mM MgCl2, 115 mM NaCl, 2.5 μM transducin, 0.01% DM, and 5 nM pigment (as determined from the extinction coefficient).

### IV. Estradiol and Related Allosteric Modulators of Opsin Signaling

In the practice of the methods of the invention, estradiol and other estradiol derived allosteric modulators of opsin signaling can all be employed. In some preferred embodiments, 17b-estradiol or an analog or derivative compound is employed. Estradiol (E2 or 17β-estradiol, also oestradiol) is a sex hormone. Estradiol is abbreviated E2 as it has two hydroxyl groups in its molecular structure. Estrone has one (E1) and estriol has three (E3). Estradiol is about 10 times as potent as estrone and about 80 times as potent as estriol in its estrogenic effect. Except during the early follicular phase of the menstrual cycle, its serum levels are somewhat higher than that of estrone during the reproductive years of the human female. Thus it is the predominant estrogen during reproductive years both in terms of absolute serum levels as well as in terms of estrogenic activity. During menopause, estrone is the predominant circulating estrogen and during pregnancy estriol is the predominant circulating estrogen in terms of serum levels. Estradiol is also present in males, being produced as an active metabolic product of testosterone. The serum levels of estradiol in males (14-55 pg/mL) are roughly comparable to those of postmenopausal women (<35 pg/mL). Estradiol in vivo is interconvertible with estrone; estradiol to estrone conversion being favored. Estradiol has not only a critical impact on reproductive and sexual functioning, but also affects other organs, including the bones.

Estradiol, like other steroids, is derived from cholesterol. After side chain cleavage and using the delta-5 or the delta-4 pathway, androstenedione is the key intermediary. A fraction of the androstenedione is converted to testosterone, which in turn undergoes conversion to estradiol by an enzyme called aromatase. In an alternative pathway, androstenedione is aromatized to estrone, which is subsequently converted to estradiol. During the reproductive years, most estradiol in women is produced by the granulosa cells of the ovaries by the aromatization of androstenedione (produced in the theca folliculi cells) to estrone, followed by conversion of estrone to estradiol by 17β-hydroxysteroid dehydrogenase. Smaller amounts of estradiol are also produced by the adrenal cortex, and (in men), by the testes. Estradiol is not produced in the gonads only: In both sexes, testosterone is converted by aromatization to estradiol. In particular, fat cells are active precursors to estradiol, and will continue to be even after menopause.

Estradiol is also produced in the brain and in arterial walls, though it cannot be readily transferred from the circulatory system into the brain.[citation needed] However, as one of the two active metabolites of testosterone in males (the other being dihydrotestosterone), it can be produced from this hormone within the brain. Estradiol enters cells freely and interacts with a cytoplasmic target cell receptor. After the estrogen receptor has bound its ligand, estradiol can enter the nucleus of the target cell, and regulate gene transcription, which leads to formation of messenger RNA. The mRNA interacts with ribosomes to produce specific proteins that express the effect of estradiol upon the target cell. Estradiol binds well to both estrogen receptors, ERα, and ERβ, in contrast to certain other estrogens, notably medications that preferentially act on one of these receptors. These medications are called selective estrogen receptor modulators, or SERMs. Estradiol is the most potent naturally occurring estrogen.

Other than 17b-estradiol, structural analogs or functional derivative compounds of estradiol may also be employed in the practice of the present invention. For example, some embodiments of the invention can employ estradiol analog compounds such as estriol, estradiol monobenzoate, estradiol 17 beta-cypionate and estradiol 3-benzoate. In some other embodiments, the estradiol molecule may be linked to an alkyl group at C17 (sometimes also at C3) position to facilitate the administration. Such modifications give rise to estradiol acetate and estradiol cypionate. Still some other embodiments of the invention employ other 17b-estradiol derivative or analog compounds, e.g., 17α-(azidopropargyl)-3,17β-estradiol and 17α-(5-azido-pent-1-ynyl)-3,17β-estradiol, ethinylestradiol or 17α-ethynylestradiol, and estradiol hemihydrate. Ethinylestradiol is the most common estrogen ingredient in combined oral contraceptive pills, is a more profound alteration of the estradiol structure. Estradiol hemihydrate, or oestradiol hemihydrate, is the hemihydrate form of estradiol. In terms of activity and bioequivalence, estradiol and its hem ihydrate are identical, with the only disparities being an approximate 1% difference in potency by weight (due to the presence of water molecules in the hemihydrate form of the substance) and a slower rate of release with certain formulations of the hemihydrate. Structures, synthesis, and pharmaceutical properties of these and other known estradiol analogs or derivatives are well documents in the art. See, e.g., Kasiotis et al., Steroids, 71: 249-255, 2006; and Sneader, Walter (2005); “Hormone analogues”. Drug discovery: a history. Hoboken, N.J.: John Wiley & Sons. pp. 188-225.

17β-estradiol and derivative compounds described herein can be obtained as medications from drug companies or de novo synthesized via routinely practiced protocols. These compounds can be easily formulated for administration in practicing methods of the invention. For example, estradiol and derivative compounds are available commercially as estradiol (Estrace), estradiol hemihydrate (Estrofem), estradiol acetate (Femtrace), estradiol valerate (Estrofem, Progynova). For de novo synthesis, 17b-estradiol can be synthesized according to various methods as described in the art. See, e.g., Wilds et al., J. Am. Chem. Soc., 68: 2125-2133, 1946; Kametani et al., J. Am. Chem. Soc., 100: 6218-6220, 1978; and Collins et al., Tetrahedron Letters, 36: 4467-4470, 1995. Estradiol methyl ethers can be synthesized via the method described in, e.g., Posner et al., J. Am. Chem. Soc., 108: 1239-1244, 1986. Ethinylestradiol can be synthesized from estron in one step as described in, e.g., Kleemann et al., Pharmaceutical Substances (5^{th} ed., Thieme-Verlag Stuttgart, 2009).

### V. Enhancing Visual Function and Treating Ocular Disorders

The invention provides methods for preserving or improving visual function in subjects or patients with symptoms or visual difficulties that are associated with or developed as a result of an ocular disease or disorder, esp. ocular degenerative disorder. In related embodiments, the invention provides methods for treating ocular degenerative disorders such as AMD by enhancing visual function and improving vision of the eyes of subjects afflicted with or at risk of developing such disorders. The methods can be used for either prophylaxis or treatment of subjects suffering from any of these visual disorders or difficulties. For example, subjects can be treated for symptoms such as impaired central vision, diminished or loss of night vision, impaired or loss of contrast sensitivity, distorted vision or blurred vision. Some other methods of the invention are directed to preventing the loss or death of photoreceptor cells in the eyes of subjects afflicted with or at risk of developing an ocular degenerative disorder. The therapeutic methods of the invention typically involve administering (via a local route or a systemic route described herein) to a subject in need of treatment a pharmaceutical composition that contains a therapeutically effective amount of an estradiol or derivative compound. In some embodiments, the compound is topically administered to one or more eyes of the subject, e.g., via eye drops.

The subjects suitable for treatment with methods of the invention can be neonatal, juvenile or fully mature adults. In some embodiments, the subjects to be treated are neonatal subjects suffering from ocular degenerative disorders such as oxygen induced retinopathy or retinopathy of prematurity. The subject to be treated may be at risk of developing the disorder (e.g., AMD), may present with one or more symptoms of the disorder, and/or may be already undergoing therapy for the disorder using other therapies, either singly or in combination. For example, subjects suitable for methods of the invention can be one who undergoes prophylactic laser treatment of bilateral drusen (Ophthalmology 105:11-23, 1998). The regimen described herein can also be combined with various treatments for subjects already diagnosed with AMD, e.g., phototherapy via targeting light to the macular area containing the lesion of nascent defective blood vessels to inhibit or impair their function (see, e.g., Archives of Ophthalmology, 119: 198-207, 2001). The treatment regimen of the invention can also be used in combination with various other therapeutic agents described below for improving visual function or for treating symptoms associated with ocular disorders.

Diseases or disorders suitable for treatment with methods of the invention include any ocular diseases and disorders that can lead to or associated with retinal aging, retinal degeneration or ocular neovascularization. These include various ocular vascular disorders (e.g., retinal neovascularization), retinal degenerative diseases (e.g., atrophic macular degeneration), retina edema (including macular edema), ischemic retinopathies, vascular hemorrhages, vascular leakage, choroidopathies, retinal injuries and retinal defects involving an interruption in or degradation of the retinal vasculature. Specific examples include but are not limited to macular degeneration (including both wet and/or dry forms), retinitis pigmentosa, diabetic retinopathy (DR), presumed ocular histoplasmosis (POHS), retinopathy of prematurity (ROP), sickle cell anemia, rod-cone dystrophies, genetic retinal degenerations (e.g., Lebers Congenital Amaurosis), and other retinal degenerations and/or dystrophies (e.g., Stargardt's disease).

In some embodiments, methods and compositions described herein are employed to provide therapeutic and/or prophylactic benefits to subjects going through normal aging or subjects suffering from age related macular degeneration, e.g., preserving vision in such subjects. In the early stages of this condition, subjects will have increased difficulty in reading and blurred vision, decreased visual sensitivity, loss of contrast sensitivity and poor dark adaptation. There two types of AMD. Dry (or atrophic) AMD is the most common type of macular degeneration and affects 90% of the people who have the condition. In the dry form, there is a breakdown or thinning of the layer of retinal pigment epithelial cells (RPE) in the macula. These RPE cells support the light sensitive photoreceptor cells that are so critical to vision. Wet macular degeneration is the less common but more severe type of AMD. With this type, the membrane underlying the retina thickens, then breaks. The oxygen supply to the macula is disrupted and the body responds by growing new, abnormal blood vessels. These begin to grow through the breaks of the membrane behind the retina towards the macula, often raising the retina. These abnormal blood vessels tend to be very fragile. They often grow, leak or bleed, causing scarring of the macula. This fluid is called exudate and wet AMD is sometimes called exudative macular degeneration.

Therapeutic or prophylactic methods of the invention can be used in subjects with either dry AMD or wet AMD. Some methods of the inventive are directed to provide prophylactic benefits to subjects by delaying the onset or severity of symptoms of AMD. The methods of the inventive can be directed to improving visual function improving visual sensitivity, Some embodiments of the invention are directed therapeutic methods for enhancing visual function in subject with AMD, e.g., via improving visual sensitivity or visual acuity, improving contrast sensitivity and dark adaptation, preventing or reverse further vision loss and blurred vision, and/or reducing the need for retreatments. Methods and compositions of the invention can be used to prevent, stabilize, reverse and/or treat macular degeneration or visual acuity loss. The methods allows reducing the risk of developing late stage or advanced age-related macular degeneration in subjects with early age-related macular degeneration, and/or by reducing the risk of vision loss associated with the development of cataracts. In some embodiments, methods of the invention are directed to treating subjects with the dry form of (or atrophic) of AMD. Currently there is no effective treatment that improves vision for patients with this form of AMD. Some other embodiments of the invention are directed to enhancing visual function in subjects with the angiogenic or wet AMD.

In some embodiments, the invention provides methods for preserving and improving visual function in subjects with various other ocular degenerative diseases other than macular degeneration. These include various other retinal degenerative diseases or ocular vascular diseases. Some methods are directed to enhancing visual function in subjects with diseases associated with retinal/choroidal neovascularization. Such diseases include, but are not limited to, diabetic retinopathy, sickle cell anemia, sarcoid, syphilis, pseudoxanthoma elasticum, Pagets disease, vein occlusion, artery occlusion, carotid obstructive disease, chronic uveitis/vitritis, mycobacterial infections, Lyme's disease, systemic lupus erythematosis, retinopathy of prematurity, Eales disease, Bechets disease, infections causing a retinitis or choroiditis, presumed ocular histoplasmosis, Bests disease, myopia, optic pits, Stargarts disease, pars planitis, chronic retinal detachment, hyperviscosity syndromes, toxoplasmosis, trauma and post-laser complications.

### VI. Pharmaceutical Compositions and Administration

The invention provides pharmaceutical compositions or formulations as well as physical devices or combinations for carrying out the therapeutic methods described herein. To enhance visual function or improve visual sensitivity, estradiol or a derivative compound described herein is administered to a subject in need of treatment at a dosage that is therapeutically effective. Typically, the active ingredient estradiol (or an analog or derivative compound) is provided in a pharmaceutical composition. In addition to estradiol or a derivative compound, the pharmaceutical composition to be administered to the subjects can also include a pharmaceutically acceptable carrier or vehicle, which can be, e.g., salt, solvate, hydrate, stereoisomer, clathrate, or prodrug thereof. In combination therapy, the pharmaceutical composition can additionally contain a therapeutically or prophylactically effective amount of a second active agent. The second active agents include any conventional therapeutics used to treat or prevent ocular degenerative disorders like macular degeneration. These include steroids, light sensitizers, integrins, antioxidants, interferons, xanthine derivatives, growth hormones, neutrotrophic factors, regulators of neovascularization, anti-VEGF antibodies, prostaglandins, antibiotics, phytoestrogens, anti-inflammatory compounds and antiangiogenesis compounds, and other therapeutics as described in, e.g., the Physicians' Desk Reference, PDR Network, LLC (2012). Specific examples of second active agents include, but are not limited to, verteporfin, purlytin, an angiostatic steroid, rhuFab, interferon-2α, and pentoxifylline.

For both therapeutic and prophylactic applications, the pharmaceutical compositions of the invention may be administered to the subject via either a local route or a systemic route. In some embodiments, local administration of the composition is desired in order to achieve the intended therapeutic effect. Thus, the pharmaceutical compositions of the present invention can be in a form suitable for an ocular route of administration. Examples of such routes include topical application, intravitreal, intraocular (intracameral), subconjunctival, sub-Tenon's, and retrobulbar injections. In some preferred embodiments, the pharmaceutical composition is topically administered to one or more eyes of the subject as eye drops. Other than the active ingredient, the topical drop composition can optionally further contain a tonicity agent, a pH adjuster, a preservative, a demulcent, a buffering agent, a lubricant, or combinations thereof. In some other embodiments, the pharmaceutical formulation can be administered via intraocular injection, periocular injection, or intravitreal injection. The injectable pharmaceutical formulation of the present invention is ophtalmically acceptable, i.e., it is appropriate for administration directly into the eye including aqueous and vitreous humors. Injectable pharmaceutical formulations can be sterile suspensions, solutions or emulsions in aqueous or oily vehicles. In some other embodiments, a systemic route may be employed for administering the pharmaceutical compositions of the invention (e.g., parenteral, enterical or oral administration).

Pharmaceutical compositions of the invention can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. For example, topical formulations can be are prepared with the estradiol compound and an ophthalmically acceptable tonicity agent. The tonicity agent is used to adjust the salt concentration include dextrose, sodium chloride, calcium chloride, potassium chloride, magnesium chloride, and boric acid. Additionally, one or more pH adjusting agents can be used in the topical formulations to adjust the pH of the composition to the desired level. Examples of pH adjusting agents include hydrochloric acid, acetic acid, sodium hydroxide, potassium hydroxide, an alkali earth metal hydroxide, an alkaline earth metal hydroxide, an organic base, an organic acid, and combinations thereof. The topical formulations can further include a preservative to keep the active ingredient chemically stable and to inhibit the growth of microorganisms in the composition. Suitable preservatives include, e.g., α-lipoic acid, α-tocopherol, ascorbyl palmitate, benzyl alcohol, biotin, bisulfites, butylated hydroxyanisole (BHA), butylated hydroxytoluene (BHT), ascorbic acid and its esters, carotenoids, calcium citrate, acetyl-L-carnitine, chelating agents, chondroitin, citric acid, coenzyme Q-IO, cysteine, cysteine hydrochloride, 3-dehydroshikimic acid (DHS), EDTA (ethylenediaminetetraacetic acid) and salts thereof, ferrous sulfate, folic acid, fumaric acid, niacin, tocopherol and their esters, e.g., tocopherol acetate, tocopherol succinate, tocotrienal, d-α-tocopherol acetate, vitamin A and its esters, vitamin B and its esters, vitamin C and its esters, vitamin D and its esters, vitamin E and its esters, e.g., vitamin E acetate, and combinations thereof. Finally, the topical eye drop formulation can include a demulcent which, when applied to an ocular surface, can lubricate, soothe and/or protect the mucous membrane of the eye. Examples of suitable demulcents include water soluble cellulose derivative, polyethylene glycol, polyvinyl alcohol, dextran, gelatin, polyol, polyvinyl alcohol, povidone, chondroitin sulfate, carbomer, tragacanth, hyaluronic acid, colloidal magnesium aluminum silicate, sodium alginate, and combinations thereof.

For pharmaceutical compositions used in other routes of administration, there are various suitable carriers that can be used to provide parenteral dosage forms of the invention. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water-miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate. Compounds that increase the solubility of one or more of the active ingredients disclosed herein can also be incorporated into the parenteral dosage forms of the invention.

The pharmaceutical compositions can be formulated for a single, one-time use or can be formulated in multiple doses. The components of a combination therapy can be in the same or different pharmaceutical compositions and can be administered simultaneously or sequentially. In certain embodiments, the pharmaceutical formulation can be lyophilized in sterile bottles or in sterile pre-filled syringes or sterile pre-filled bags which can be frozen or refrigerated. The formulation can also be lyophilized and reconstituted with a suitable carrier or vehicle. The selected dosage level depends upon a variety of pharmacokinetic factors including the activity of the particular compositions of the present invention employed, the route of administration, the time of administration, and the rate of excretion of the particular compound being employed. It also depends on the duration of the treatment, other drugs, compounds and/or materials used in combination with the particular compositions employed, the age, gender, weight, condition, general health and prior medical history of the subject being treated, and like factors. Methods for determining optimal dosages are described in the art, e.g., Remington: The Science and Practice of Pharmacy, Mack Publishing Co., 20^{th} ed., 2000. As general guidance, a daily dosage of composition can be expected to be from about 0.001 mg to about 100 mg, from about 0.01 mg to about 10 mg, or from about 0.1 mg to about 1 mg per kilogram of body weight. When the composition is a liquid, the per-eye volume of the composition to be administered should in general be less than about 10 mL, e.g., from about 0.05 mL to about 5 mL, from about 0.1 mL to about 3 mL, or from about 0.2 mL to about 1 mL. When administered as eye drops, the amount to be administered can vary, e.g., 1 to 20 drops, 1 to 10 drops, or 2 to 5 drops. The preferred daily dose can be split and administered over several times a day (e.g., once per day, twice per day, three times per day, or more). Also, the dosage and frequency of administration can vary depending on whether the treatment is prophylactic or therapeutic. In prophylactic applications, a relatively low dosage may be administered at relatively infrequent intervals over a long period of time. Some subjects may continue to receive treatment for the rest of their lives. In therapeutic applications, a relatively high dosage at relatively short intervals may be required until progression of the disease is reduced or terminated, and preferably until the subject shows partial or complete amelioration of symptoms of the ocular vascular disease. Thereafter, the subject can be administered a prophylactic regime.

The invention further provides pharmaceutical combinations (e.g., kits) for carrying out the various therapeutic applications described herein. Such pharmaceutical combination can contain an estradiol compound or functional derivative disclosed herein, in free form or in a composition (e.g., a topical eye drop formulation), an optional co-agent or carrier, a suitable device or applicator for ocular administration of the pharmaceutical composition (e.g., eye drop dispenser), as well as instructions for administration of the agents to enhance visual function and to treat subjects with retinal degenerative disorders. The kits can also include a container for storing the components of the kits. The container can be, for example, a bag, box, envelope or any other container that would be suitable for use in the present invention.

### EXAMPLES

The following example is provided to further illustrate the invention but not to limit its scope.

### Example 1

### Estradiol Enhances Opsin Signaling and Transducin Activation

We examined the effect of the 17β-estradiol on opsin signaling and transducin activation. We measured the ability of estradiol to modulate rhodopsin activation of bovine rod transducin using the radioactive filter binding assay as described in Kono et al., Biochemistry 44:799-804, 2005; and Kono & Crouch, Methods Mol. Biol. 652:85-94, 2010. Specifically, bovine rhodopsin is illuminated with a 15 sec pulse of yellow light and the binding of 35S-GTPγS to transducin is measured in the presence (Rho+estradiol) and absence (Rho alone) of estradiol. At each time point, the reaction was stopped, e.g., by a 1:100 dilution in buffer containing an excess of cold GTPγS, followed by filtering and washing through a nitrocellulose membrane on a vacuum manifold. The proteins, including transducin and bound 35S-GTPγS, adhere to the membrane and the unbound GTPγS flows through. The bound 35S-GTPγS on the filter membranes is counted and converted to pmol GTPγS.

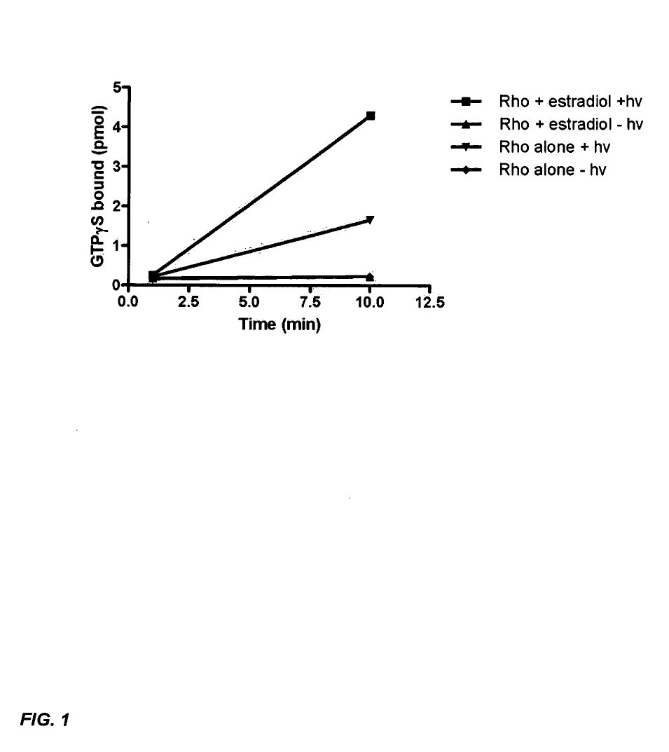

As shown in FIG. 1, transducin binds about three-fold more 35S-GTPγS in the presence of 17β-estradiol than in the absence of 17β-estradiol. The same study has been performed in multiple independent experiments which yielded similar results.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, GenBank sequences, patents, and patent applications cited in this specification are herein incorporated by reference as if each was specifically and individually indicated to be incorporated by reference.

What is claimed is:

1. A method of enhancing visual function and improving vision in a subject, comprising administering to one or more eyes of the subject a pharmaceutical composition comprising a therapeutically effective amount of an estradiol or derivative compound, thereby enhancing visual function and improving vision of the subject.

2. The method of claim 1, wherein the administered estradiol or derivative compound is 17β-estradiol.

3. The method of claim 1, wherein the estradiol or derivative compound is locally administered to the eyes.

4. The method of claim 1, wherein the estradiol or derivative compound is locally administered via eye drops.

5. The method of claim 1, wherein the pharmaceutical composition further comprises a pharmaceutically acceptable carrier.

6. The method of claim 1, wherein the subject is afflicted with or at risk of developing an ocular degenerative disorder.

7. The method of claim 6, wherein the ocular degenerative disorder is a retinal degenerative disorder or ocular vascular disorder.

8. The method of claim 6, wherein the ocular degenerative disorder is age-related macular degeneration (AMD).

9. A method of preserving vision in the eyes of a subject, comprising prophylactically administering to one or more eyes of the subject a pharmaceutical composition comprising a therapeutically effective amount of an estradiol or derivative compound, thereby preserving vision and preventing the loss or death of photoreceptor cells in the subject.

10. The method of claim 8, wherein the estradiol or derivative compound is 17β-estradiol.

11. The method of claim 8, wherein the estradiol or derivative compound is locally administered to the eyes.

12. The method of claim 8, wherein the estradiol or derivative compound is locally administered via eye drops.

13. The method of claim 8, wherein the pharmaceutical composition further comprises a pharmaceutically acceptable carrier

14. The method of claim 8, wherein the subject is afflicted with or at risk of developing an ocular degenerative disorder.

15. The method of claim 8, wherein the ocular degenerative disorder is a retinal degenerative disorder or ocular vascular disorder.

16. The method of claim 8, wherein the ocular degenerative disorder is age-related macular degeneration (AMD).

17. A method of improving or preserving vision of a subject afflicted with or at risk of developing age-related macular degeneration (AMD), comprising administering to the subject a pharmaceutical composition comprising a therapeutically effective amount of an estradiol or derivative compound, thereby improving or preserving vision of the subject.

18. The method of claim 17, wherein the estradiol or derivative compound is 17β-estradiol.

19. The method of claim 17, wherein the estradiol or derivative compound is locally administered to one or more eyes of the subject.

20. The method of claim 17, wherein the estradiol or derivative compound is locally administered via eye drops.
